(19) United States
(12) Patent Application Publication
(10) Pub. No.: US 2004/0006371 A1
(43) Pub. Date: Jan. 8, 2004

(54) Method for reducing mandibular angle fractures and forceps used for the method

(76) Inventor: Byoung Ho Choi, Wonju (KR)

Correspondence Address: PENNIE AND EDMONDS, 1155 AVENUE OF THE AMERICAS, NEW YORK, NY, 100362711

(21) Appl. No.: 10/381,117

(22) PCT Filed: Mar. 2, 2001
(86) PCT No.: PCT/KR01/00314

(51) Int. Cl. A61B 17/28

(52) U.S. Cl. 606/205

(57) ABSTRACT

The invention relates to a method for reducing mandibular angle fractures and forceps used for the method.

The method comprises of forming a pressing point 18 on the outer side of the proximal fragment 13 and a pressing point 19 on the inner side or the upper side of the distal fragment 15 respectively, and the step of applying a compressing force to the pair of pressing points 18 and 19. The prongs of the forceps comprise of the linear portions 25 and 35 and bending portions 28 and 38 extending from the front ends of the linear portions 25 and 35. The bending portions are curved to fit into an engagement hole 18 on the outer side of the proximal fragment 13 and an engagement hole 19 on the inner or upper side of the distal fragment 15 respectively via a transoral approach.

### FIELD OF THE INVENTION

The invention relates to a method for reducing mandibular angle fractures and forceps used for the method. In particular, it relates to a method for reducing mandibular angle fractures in which surgical fixation of mandibular fractures can be easily conducted on the fractured site in a mandibular angle area via a transoral approach by reducing the displaced proximal and distal fragments to a normal position correctly. In addition, the invention describes a set of forceps that can be easily introduced to the fractured site in a mandibular angle area via a transoral approach, and can be used to compress and hold the fractured site firmly together to achieve accurate reduction and stable fixation of the fracture.

### BACKGROUND OF THE INVENTION

In cases of mandibular angle fractures, there are many problems due to the lack of appropriate forceps and proper methods for reducing mandibular angle fractures. In cases of mandibular angle fractures, the most currently used method of fixation is the application of a metal plate across the fracture at the superior border of the mandible via a transoral approach. As the metal plate can be placed via an intraoral approach, several advantages are offered. Facial scarring is minimized, and there is a lesser likelihood of damage to both the facial nerve and the inferior alveolar nerve. In order to achieve an accurate reduction and stable fixation of the fractures with the method of fixation, it is important to achieve a correct reduction of fractured fragments prior to applying the metal plate. It is easy to perform reduction and fixation of fractures in the area of the mandibular symphysis where its shape is convex, as this fracture can be easily reduced with the aid of conventional reduction forceps. The forceps are composed of a pair of symmetrical prong members and clicks on their handle. The forceps can hold large abutting segments of bone together and manipulate the segments into reduction. Once reduced, they can compress the fracture site and the position of the segments can be held with the locking mechanism on the handle of the forceps. However, the forceps cannot be applied in the mandibular angle region when attempted via a transoral approach. The curvature of the mandibular surface in the mandibular angle region is concave proximal to the fracture and becomes flat along the lateral aspect of the mandible distal to the fracture. The curvature is so complex that applying conventional reduction forceps at the multicurved surface is difficult. Therefore, it is not easy to perform the reduction in mandibular angle fractures. Assuming surgical fixation of mandibular angle fractures through the oral cavity by reducing the proximal and distal fragments 13 and 15 that is displaced relative to the fracture 11 by using conventional forceps, as depicted in FIG. 1, the prong members have to be engaged either with each of the pressing points 18 and 19a formed on the outer side or with each of the pressing points 18a and 19 formed on the upper side of the mandibular angle area. In that case, the fracture gap on the side opposite to the pressing points tends to open at the site of the fracture 11. This is because the prong members of the forceps press the points of the same side of mandibular angle area where the curvature of its surface is concave or flat. Moreover, the symmetrical prongs of the conventional forceps are unable to grip the points of the outer surface or the upper surface of the mandibular angle. Even though the forcep prongs can grip the points, they are easily slipped off the points when compression is applied. Conclusively, conventional forceps can not be applied intraorally for reducing mandibular angle fractures.

Therefore, in the conventional art, the reduction of mandibular angle fractures via a transoral approach has been carried out manually or with a bone-holding clamp. Grasping the proximal fragment and pulling it into reduction against the anterior fragment is the general procedure. However, it is difficult to compress or hold the fracture site together firmly before a metal plate and screws are placed. This can result in unstable fixation and probable failure. Moreover, due to the improper reduction and fixation of the fracture after the operation, patients' jaw needs to be immobilized for quite a long time until the fracture is healed to ensure stability of the fracture.

For an effective fracture cure it is important to achieve intraoperative anatomic alignment and stable fixation of the fracture. The key for this is to keep the proximal and distal fragments 13 and 15 in tight contact by correctly reducing, compressing and holding the proximal and distal fragments 13 and 15 prior to metal plate application. This might be one reason why to date, of all mandibular fractures, mandibular angle fractures are associated with the highest incidence of complications, which include postsurgical infection or non-union. It is the angle region where its thickness is thin and the powerful muscles attached to the ramus transfer their force to the body of the mandible. This creates great demands on fixation and stability. Therefore, many surgeons are currently using immobilization of jaw to ensure stability. Thereby the patients have difficulty in normal social activity during the period of jaw immobilization because of the difficulties in chewing or pronouncing as well as oral hygiene.

On the other hand, another reduction method has been developed and used in which the skin on the underside of the jaw is incised, and the approach to the fracture site is made through the incision to drive respective fixing pins in the proximal fracture fragment and the distal fracture fragment. Subsequently, the proximal fracture fragment and the distal fracture fragment are pressed using forceps so that they adhere tightly. However, as this method is performed via an extraoral approach, there are several disadvantages. Scar remains on the face after the operation, operating time increased, and operation becomes complicated with preparation through various soft tissue layers.

### SUMMARY OF THE INVENTION

The invention was created to resolve the problems with the conventional art as described above. The object of the invention is to provide a method for reducing mandibular angle fractures via a transoral approach which allows for reducing the proximal and distal fragments that are displaced due to the fracture of mandibular angle. Another object of the invention is to provide a set of forceps that can be easily introduced to the fracture site through the mouth with the ability to compress the fracture. Excellent adaptation and good stability at the fracture site can be achieved using this method and the forceps. Consequently, the infection or non-union rate following an operation is markedly improved, patients' jaws are not immobilized, and no facial scars are left. Another object of the invention is to permit good visualization and easy instrument manipulation for metal plate application, and thereby to offer the advantage of rapid, time-saving reduction and fixation of the angle fractures.

According to the first aspect of the invention, a method is provided for reducing mandibular angle fractures 11, which is fractured into the proximal fragment 13 and distal fragment 15. The method comprises of forming a pressing point 18 on the outer side of the proximal fragment 13 and a pressing point 19 on the inner side or the upper side of the distal fragment 15 respectively, and the step of applying a compressing force to the pair of pressing points 18 and 19. The line of force action passes close to the central zone of the cross section of the fracture 11 so that the entire border of the fracture 11 can be tightly contacted and compressed.

According to the second aspect of the invention, a set of forceps is provided for engagement with mandibular angle on both sides of a fracture 11. The forceps are consisted of the first member 20 and the second member 30 being combined with each other at the hinge 23 of the middle part and having grips 21 and 31 on one side and prongs 24 and 34 on the other side. Wherein the prongs 24 and 34 of the members 20 and 30 comprise of the linear portions 25 and 35 proximal to the hinge 23 and bending portions 28 and 38 extending from the front ends of the linear portions 25 and 35, the bending portions 28 and 38 are curved to fit into an engagement hole (pressing point) 18 on the outer side of the proximal fragment 13 and an engagement hole (pressing point) 19 on the inner or upper side of the distal fragment 15 respectively via a transoral approach.

According to the third aspect of the invention, said bending portion 28 of the first member 20 comprises a tip 27 being bent approximately in the same direction of the axis of the hinge 23 and extending toward the inside of mandibular angle. The said bending portion 38 of the second member 30 is comprised of an extending part 36 extending approximately in the same direction of the tip 27 of the first member 20 and a tip 37 extending from the front end of the extending part 36 substantially towards the same direction as the linear portion 25 of the first member 20. The point ends of the tip 27 and 37 of the first and second members 20 and 30 extend approximately in the opposite direction to face each other.

According to the fourth aspect of the invention, the bending portion 28 and 38 of the first and second members 20 and 30 comprises the extending parts 26 and 36 that are bent laterally from the front end of the respective linear portions 25 and 35 towards approximately the same direction of the axis of the hinge 23. The tips 27 and 37 are bent from the respective extending parts 26 and 36 towards each other in the direction approximately perpendicular to the axis of the hinge 23.

According to the fifth aspect of the invention, the prong 34 of the second member 30 extends longer than the prong 24 of the first member 20 so that the tip 37 of the second member 30 may engage with a deeper position of mandibular angle than the tip 27 of the first member 20.

### BRIEF DESCRIPTION OF THE DRAWINGS

FIG. 1 shows the conceptional view illustrating the conventional method for reducing mandibular angle fractures,

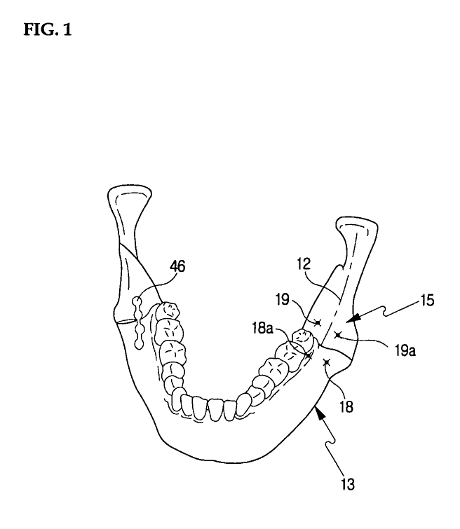

FIG. 2 shows the schematic plan view for a method of reducing mandibular angle fractures according to the invention,

FIG. 3 shows the perspective view for a method of reducing mandibular angle fractures according to the invention,

FIG. 4 shows the perspective view of an example of the forceps according to the invention,

FIG. 5 shows the perspective view illustrating the use of the forceps shown in FIG. 4,

FIG. 6 shows the perspective view of another example of the forceps according to the invention, and

FIG. 7 shows the perspective view illustrating the use of the forceps shown in FIG. 6.

### DETAILED DESCRIPTION OF THE INVENTION

Preferred embodiments of the present inventions are described in detail below in conjunction with the accompanying drawings.

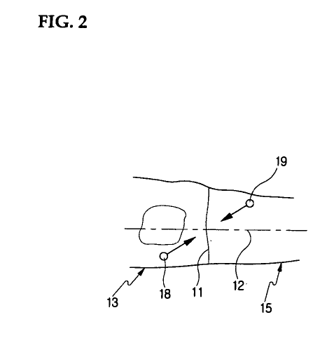

FIG. 2 is a schematic plan view of the fractured mandibular angle showing the concept of the reduction method according to the invention. As shown in FIG. 2, the compression forces are applied at a pressing point 18 on the outer surface of the proximal fragment 13 and at a pressing point 19 on the inner surface of the distal fragment 15 in the direction facing to each other, so that the opposite forces depicted by the arrow may be parallel or at the least substantially coincide with each other. In particular, when the pressing forces from the outer area on the proximal fragment 13 and from the inner area on the distal fragment 15 are applied in the direction of the arrows, the force acting lines will pass nearby the central zone of the fracture 11 cross section. Thereby the entire border of the fracture 11 can be tightly contacted and compressed. Moreover, numerous micro irregularities on the fracture section may prevent the fragments 13 and 15 from slipping. Furthermore, if the pressing points 13 and 15 are drilled by a proper instrument to form the engaging holes, the forceps will not slip from the pressing points 13 and 15.

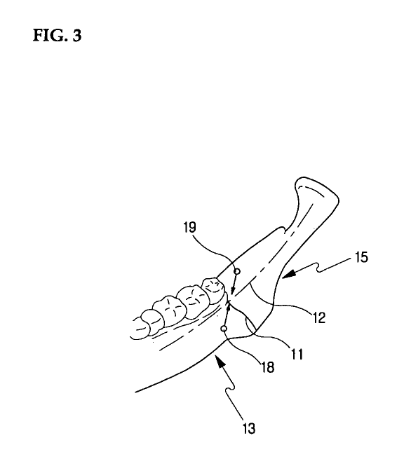

Alternatively, as shown in FIG. 3, the reduction can be achieved by applying forces via a transoral approach at the pressing point 18 on the outer area on the proximal fragment 13 and at the pressing point 19 on the upper area on the distal fragment 15 in the opposite direction facing each other. In this case, force acting lines against the two fragment 13 and 15 will also pass through the cross section of the fracture 11. This is so the proximal and distal fragments 13 and 15 can be firmly compressed to allow a proper reduction.

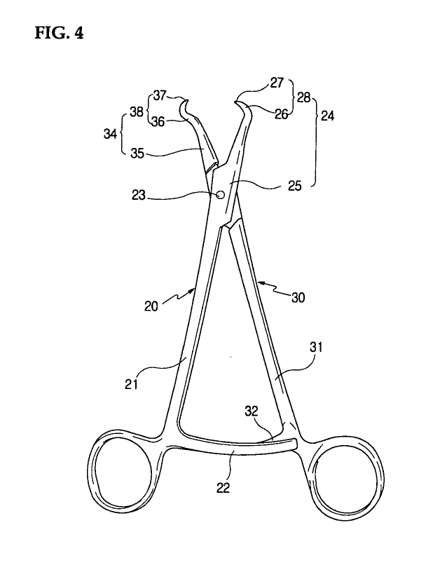

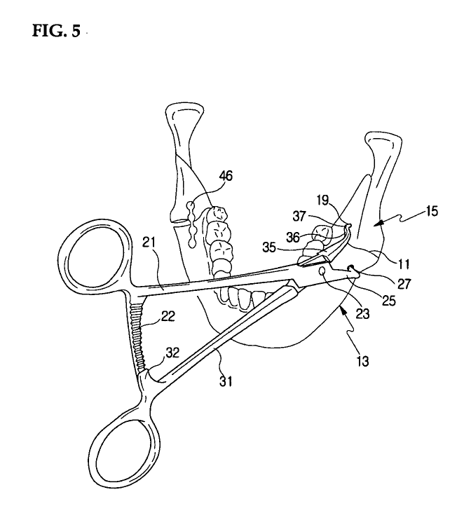

Preferable examples of forceps to realize the above reduction method are described in detail with reference to FIGS. 4 to 7. The forceps shown in FIG. 4 and FIG. 5 are used in the case that the pressing points 18 and 19 are formed on an outer position of proximal fragment 13 and on an upper position of distal fragment 15 respectively. The forceps are composed of the first member 20 and the second member 30, which are combined at the hinge 23 on the middle part. The forceps have grips 21 and 31 on one side of the member 20 and 30 and prongs 24 and 34 on the other side of the member 20 and 30. The prongs 24 and 34 of the member 20 and 30 comprise the linear portions 25 and 35 proximal to the hinge 23 and the bending portions 28 and 38 extending from the front ends of the linear portions 25 and 35.

The tip 27 of the bending portion 28 is curved to extend approximately laterally or parallel to the axis of the hinge 23 so that it may engage via a transoral approach with the hole of the pressing point 18 on the outer side of the proximal fragment 13. Meanwhile, the bending portion 38 of the second member 30 comprises the extending part 36 and a tip 37. The extending part 36 extends approximately in the same direction of the axis of the hinge 23 or the tip 27 of the first member 20. The tip 27 extends from the front end of the extending part 26 substantially towards the same direction of the linear portion 25 of the first member 20. The tip 37 can be fitted in the pressing point or the engaging hole 19 at an upper and front side of the mandibular angle through the oral cavity. The tips 27 and 37 of the first and second members 20 and 30 are substantially oriented to face each other or to form an acute angle between their imaginary extension lines. This shape will prevent the tip 27 and 37 from slipping off the engaging holes 18 and 19 when compression is applied.

The tips 27 and 37 of the first and second members 20 and 30 are each bent to one side from the linear portions 25 and 35 by the extension parts 26 and 36. Therefore, the cheek flesh within the oral cavity can be pushed outward by the extending parts 26 and 36 as well as the linear portions 25 and 35 so as to permit good visualization during the surgical procedure on the bone fracture.

The numerals 22 and 32 stand for locking mechanism or click which maintain or lock the first and second members 20 and 30 in the state of clamping on proximal and distal fragments 13 and 15. The numeral 46 stands for a metal plate used to fix the fractured fragments 13 and 15 at the reduced state.

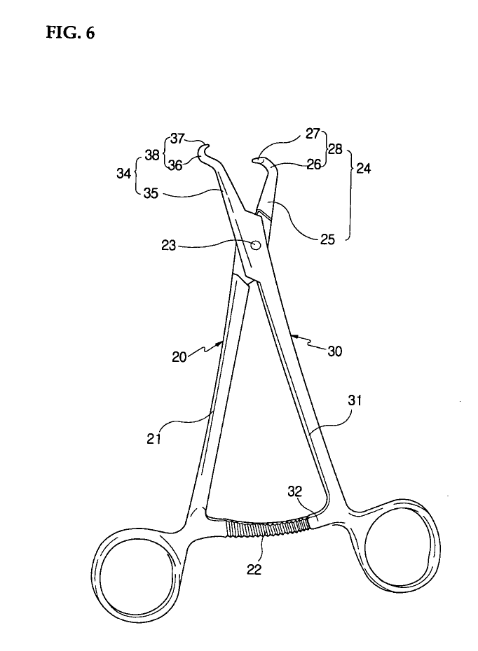

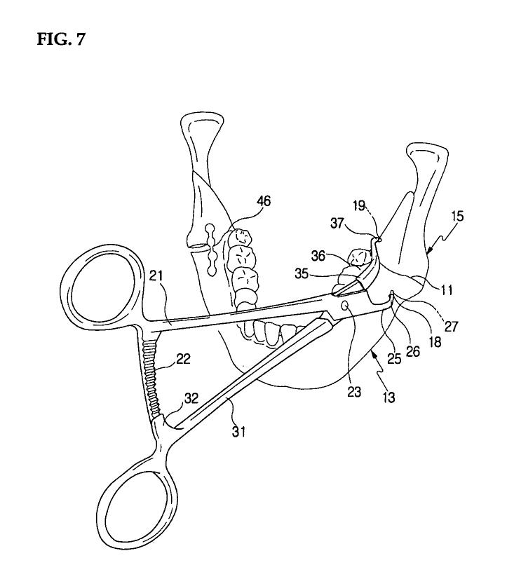

FIGS. 6 and 7 show another example of forceps according to the invention. These forceps are used in the case that the pressing points 18 and 19 are formed on an outer position of the proximal fragment 13 and on an inner position of the distal fragment 15 respectively. These forceps also include prongs 24 and 34 on the first and second members 20 and 30. The prongs 24 and 34 comprise the linear portions 25 and 35 proximal to the hinge 23 and bending portions 28 and 38 that are curved from the front ends of the linear portions 25 and 35.

The bending portions 28 and 38 are composed of the extension parts 26 and 36 and tips 27 and 37. The extending parts are bent from the front end of linear portions 25 and 35 to the direction of the axis of hinge 23 or in the lateral direction. The tips 27 and 37 are bent from the front end of the extending parts 26 and 36 towards each other in the direction approximately perpendicular to the axis of the hinge 23. As a result, the tips 27 and 37 will extend towards the imaginary center line 12 of the mandibular angle. The linear portion 35 of the second member 30 is longer than the corresponding linear portion 25 of the first member. Therefore, when the forceps are introduced to the fracture site via a transoral approach, the tip 37 on the longer linear portion 35 of the second member 30 can be naturally located at deeper position inside the oral cavity than the corresponding tip 27 of the first member 20. This will result in the easy and correct positioning of the tips 27 and 37 into the pressing point 18 on the outer position of the proximal fragment 13 and the pressing point 19 on the inner position of the distal fragment 15.

In these examples as well, as the tips 27 and 37 are bent to one side from the linear portions 25 and 35 as much as the extension parts 26 and 36, the cheek flesh can be pushed outward by the extending parts 26 and 36 as well as linear portions 25 and 35, which can permit good visualization during the surgical procedure on the bone fracture.

A method for an open reduction of mandibular angle fractures using the present invention is described.

First, the engaging holes should be drilled at the pressing points 18 and 19 on the proximal and distal fragment 13 and 15. Subsequently, the surfaces of the fracture 11 of the proximal and distal fragments 13 and 15 are combined together firmly, and in that state, the forceps as illustrated in FIGS. 4 to 7 are used. The forceps are approached to the fracture 11 through the mouth. And, the tips 27 and 37 at the front end of the first and second members 20 and 30 are to fit in the engagement holes 18 and 19 formed on the proximal and distal fragments 13 and 15. After this, the forcep grips 21 and 31 are closed together to produce the pressure required for the reduction.

In order to compress the entire border of the fracture 11 with the forceps, it is important to position the engagement holes 18 and 19 both at the outer position of the proximal fragment 13 and at the inner or upper position of the distal fragment 15. When a compression force is applied to the points 18 and 19, the line of force action passes near the central zone of the fracture 11 cross section, whereby effective compression can be conducted to achieve a good result. At that time, numerous micro irregularities on the fracture 11 play a role in preventing slippage between the proximal and distal fragments 13 and 15. Thereby they increase the compressing and fixing effect.

When the proximal and distal fragments 13 and 15 have been restored to the normal position, a metal plate 46 is then placed and fixed by screws across the fracture site, while the reduced state is being maintained by the clickers 22 and 32 of the forceps. The clickers lock up the reduced state for the two bone fragments 13 and 15. As mentioned above, as the tips 27 and 37 of the first and second members 20 and 30 are each bent to one side from the linear portions 25 and 35, the cheek flesh can be pushed outward by the linear portions 25 and 35, and permit good visualization during the work for fixing metal plates.

It is to be understood that, while the invention was described mainly with respect to a few preferable specific embodiments, the invention is never restricted to those embodiments and a variety of modifications and alterations would be possible to a man skilled in the art by referring to the description or drawings presented here and within the spirit of the invention and thus those modifications or alterations are to fall within the scope of the invention, which scope should be limited only by the attached claims.

What is claimed is:

1. A method for reducing mandibular angle fractures 11, which is fractured into the proximal fragment 13 and distal fragment 15, the method comprising of forming a pressing point 18 on the outer side of the proximal fragment 13 and a pressing point 19 on the inner side or the upper side of the distal fragment 15 respectively, and the step of applying a compressing force to the pair of pressing points 18 and 19, in such a manner the line of force action passes close to the central zone of the cross section of the fracture 11 so that the entire border of the fracture 11 can be tightly contacted and compressed.

2. A set of forceps for engagement with mandibular angle on both sides of a fracture 11, consisting of the first member 20 and the second member 30 being combined with each other at the hinge 23 of the middle part and having grips 21 and 31 on one side and prongs 24 and 34 on the other side, wherein the prongs 24 and 34 of the members 20 and 30 comprise linear portions 25 and 35 proximal to the hinge 23 and bending portions 28 and 38 extending from the front ends of the linear portions 25 and 35, the bending portion 28 of the first member 20 being curved to fit into an engagement hole (pressing point) 18 on the outer side of the proximal fragment 13 via a transoral approach and the bending portion 38 of the second member 30 being curved to fit into an engagement hole (pressing point) 19 on the inner or upper side of the distal fragment 15 through the mouth.

3. A set of forceps of claim 2 said, wherein the bending portion 28 of the first member 20 comprises the tip 27 being bent approximately in the same direction of the axis of the hinge 23 and extending toward the inside of mandibular angle, and said bending portion 38 of the second member 30 is comprised of an extending part 36 extending approximately in the same direction of the tip 27 of the first member 20 and a tip 37 extending from the front end of the extending part 36 substantially towards the same direction as the linear portion 25 of the first member 20, and the point ends of the tip 27 and 37 of the first and second members 20 and 30 extend approximately in the opposite direction to face each other.

4. A set of forceps of claim 2 said, wherein the bending portion 28 and 38 of the first and second members 20 and 30 comprises the extending parts 26 and 36 being bent laterally from the front end of the respective linear portions 25 and 35 towards approximately the same direction of the axis of the hinge 23, and tips 27 and 37 being bent from the respective extending parts 26 and 36 towards each other in the direction approximately perpendicular to the axis of the hinge 23.

5. A set of forceps of claim 2, wherein the prong 34 of the second member 30 extends longer than the prong 24 of the first member 20 so that the tip 37 of the second member 30 may engage with a deeper position of mandibular angle than the tip 27 of the first member 20.
